(19) United States
(12) Patent Application Publication
(10) Pub. No.: US 2014/0228733 A1
(43) Pub. Date: Aug. 14, 2014

(54) Left Atrial Appendage Shunt

(71) Applicant: Cook Medical Technologies LLC, Bloomington, IN (US)

(72) Inventors: Michelle D. Martinez, Winston-Salem, NC (US); Shaun Davis Gittard, Winston-Salem, NC (US); Devesh Amatya, Winston-Salem, NC (US)

(73) Assignee: Cook Medical Technologies LLC, Bloomington, IN (US)

(21) Appl. No.: 14/167,090

(22) Filed: Jan. 29, 2014

Related U.S. Application Data
(60) Provisional application No. 61/764,593, filed on Feb. 14, 2013.

(51) Int. Cl. A61M 27/00

(52) U.S. Cl. CPC A61M 27/002
USPC 604/8

(57) ABSTRACT

A left atrial appendage shunt for reducing blood clot formation in a left atrial appendage includes a first artificially created opening through a wall of the left atrial appendage and a second artificially created opening through a wall of a target cardiovascular structure. A circulation conduit fluidly connects an interior of the left atrial appendage and an interior of the target cardiovascular structure, and has a first end opening at the first artificially created opening and a second end opening at the second artificially created opening.

### TECHNICAL FIELD

The present disclosure relates generally to a left atrial appendage shunt, and more particularly to a shunt fluidly connecting an interior of the left atrial appendage and an interior of a target cardiovascular structure for reducing blood clot formation in the left atrial appendage.

### BACKGROUND

The left atrial appendage is a small muscular pouch connected to the left atrium of the heart. During normal functioning of the heart, the left atrial appendage contracts with the left atrium and acts as a reservoir for blood flowing through the atrium. In patients with atrial fibrillation, the contractions of the left atrium and the right atrium are not properly synchronized with the contractions of the left ventricle and the right ventricle. When this occurs, blood may not be fully ejected from the left atrial appendage and can pool. Additionally, the highly trabeculated structure of the left atrial appendage provides flow resistant voids where blood is more likely to pool. This pooled blood may result in blood clot formation in the left atrial appendage. If these blood clots dislodge, they may form an embolism, which can result in disability and even death through occlusion of cerebral and peripheral vasculature.

Current treatment options include the administration of blood thinning medications, occlusion of the left atrial appendage, and removal of the left atrial appendage. For example, a catheter may be used to deliver an occlusion device into the left atrial appendage, or the left atrial appendage may be surgically closed or removed. An exemplary occlusion device is provided in U.S. Patent Application Publication No. 2009/0171386 to Amplatz et al. In particular, the Amplatz et al. occlusion device may be introduced into the left atrial appendage using a delivery catheter and may include a first portion having at least one plane of occlusion that is positioned outside of the left atrial appendage, and a second portion having at least one plane of occlusion that is positioned within a cavity defined by the left atrial appendage. Although this occlusion device may be suitable for some applications, occlusion devices may be traumatic to the left atrial appendage, may become dislodged, and/or may fail to completely occlude the left atrial appendage. In addition, the anchoring mechanism of some occlusion devices may result in arrhythmias such as atrioventricular block.

The present disclosure is directed toward one or more of the problems or issues set forth above.

### SUMMARY OF THE DISCLOSURE

In one aspect, a left atrial appendage shunt for reducing blood clot formation in a left atrial appendage includes a first artificially created opening through a wall of the left atrial appendage and a second artificially created opening through a wall of a target cardiovascular structure. A circulation conduit fluidly connects an interior of the left atrial appendage and an interior of the target cardiovascular structure, and has a first end opening at the first artificially created opening and a second end opening at the second artificially created opening.

In another aspect, a method of reducing blood clot formation in a left atrial appendage using a left atrial appendage shunt includes steps of forming a first artificially created opening through a wall of the left atrial appendage and forming a second artificially created opening through a wall of a target cardiovascular structure. An interior of the left atrial appendage and an interior of the target cardiovascular structure are fluidly connected with a circulation conduit having a first end opening at the first artificially created opening and a second end opening at the second artificially created opening.

### BRIEF DESCRIPTION OF THE DRAWINGS

FIG. 1 is a cross sectional view of upper chambers of a heart, depicting a first exemplary shunt fluidly connecting a left atrial appendage and a pulmonary vein;

FIG. 2 is a perspective view of the first exemplary shunt formed as a direct anastomosis, according to one exemplary embodiment;

FIG. 3 is a perspective view of the first exemplary shunt formed as a direct anastomosis, according to another exemplary embodiment;

FIG. 4 is a perspective view of the first exemplary shunt formed using a vessel;

FIG. 5 is a partial cross sectional view of one stage of a stent placement procedure for forming the first exemplary shunt;

FIG. 6 is a partial cross sectional view of another stage of the stent placement procedure of FIG. 5;

FIG. 7 is a cross sectional view of the upper chambers of the heart shown in FIG. 1, depicting a second exemplary shunt fluidly connecting the left atrial appendage and a left ventricle;

FIG. 8 is a partial cross sectional view of a heart, depicting a third exemplary shunt fluidly connecting the left atrial appendage and a descending aorta; and

FIG. 9 is a partial cross sectional view of the heart of FIG. 8, depicting a fourth exemplary shunt fluidly connecting the left atrial appendage and a subclavian artery.

### DETAILED DESCRIPTION

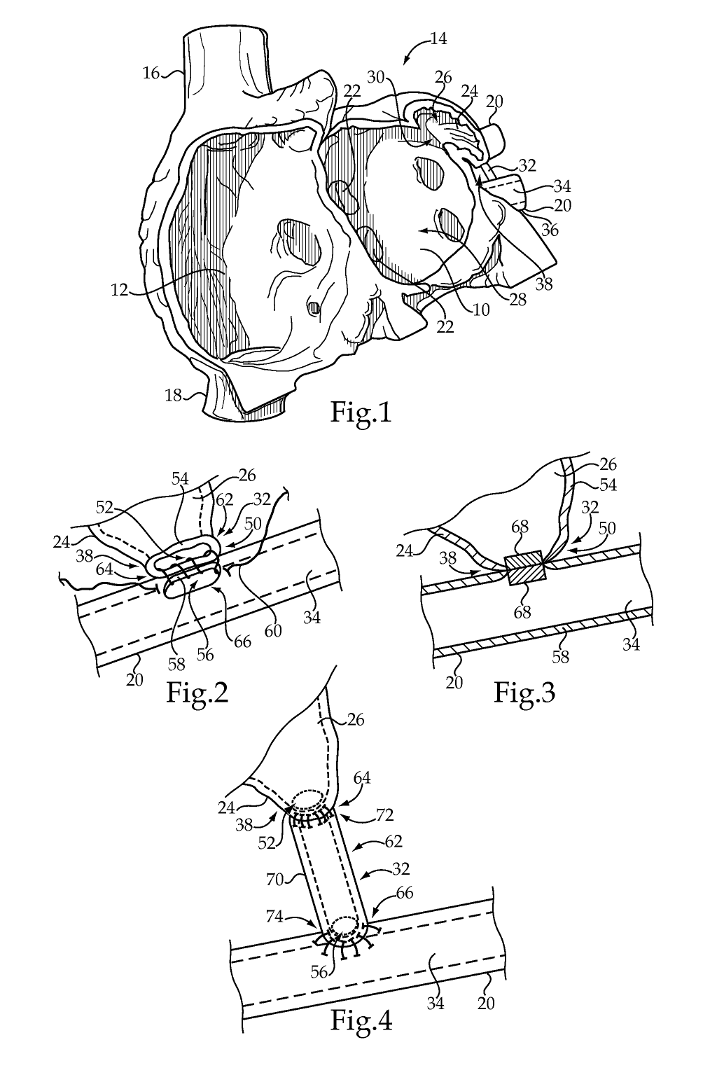

FIG. 1 depicts a left atrium 10 and a right atrium 12 of a heart 14. The atria 10 and 12 receive blood returning to the heart 14 from other areas of the body. In particular, the right atrium 12 receives de-oxygenated blood returning from a superior vena cava 16 and an inferior vena cava 18, while the left atrium 10 receives oxygen-rich blood returning to the heart 14 from the lungs via left pulmonary veins 20 and right pulmonary veins 22. Although not depicted, lower chambers of the heart 14, or left and right ventricles, function to pump blood out of the heart 14. In particular, the right ventricle receives the de-oxygenated blood from the right atrium 12 and pumps it to the lungs via a main pulmonary artery, while the left ventricle receives the oxygen-rich blood from the left atrium 10 and pumps it to the aorta, which distributes the oxygen-rich blood to the rest of the body.

A left atrial appendage 24 is a muscular pouch connected to the left atrium 10 that acts as a reservoir for the left atrium 10. The left atrial appendage 24 lies within a membrane, or pericardium, enclosing the heart 14, and has a fluid connection with the left atrium 10. In particular, an interior 26 of the left atrial appendage 24 is fluidly connected with an interior 28 of the left atrium 10 via an ostium 30 of the left atrial appendage 24. Although a particular configuration of the left atrial appendage 24 is shown, it should be appreciated that the size and shape of the left atrial appendage 24 can vary greatly from person to person.

A first exemplary embodiment of a left atrial appendage shunt for reducing blood clot formation in the left atrial appendage 24 is shown generally at 32. In particular, the left atrial appendage shunt 32 may be configured to fluidly connect the interior 26 of the left atrial appendage 24 and an interior 34 of a target cardiovascular structure 36. As shown, the left atrial appendage shunt 32 is distinct from the blood flow passage provided by the ostium 30 of the left atrial appendage 24, and may be provided at a distal tip 38 of the left atrial appendage 24. According to the first exemplary embodiment, the target cardiovascular structure 36 may include one or more of the pulmonary veins 20 and 22 or, more particularly, one or both of the left pulmonary veins 20. The left pulmonary veins 20 are located in close proximity to the left atrial appendage 24 and feed the left atrium 10.

If at least one of the pulmonary veins 20 and 22 are utilized as the target cardiovascular structure 36, when the left atrium 10 contracts, blood may flow from the left atrial appendage 24, through the left atrial appendage shunt 32, and into the selected pulmonary vein(s) 20 and/or 22. When the right ventricle contracts, blood may flow from the pulmonary vein(s) 20 and/or 22, through the left atrial appendage shunt 32, and into the left atrial appendage 24. Thus, bi-directional flow through the left atrial appendage shunt 32 is contemplated. Although a single shunt 32 is described, it should be appreciated that multiple shunts may be formed.

Turning now to FIG. 2, a first exemplary means for forming the left atrial appendage shunt 32 will be described. In particular, the left atrial appendage shunt 32 may be formed by creating a direct anastomosis 50 between the left atrial appendage 24 and one of the left pulmonary veins 20 (or other target cardiovascular structure 36). As shown, a first artificially created opening 52 may be formed through a wall 54 of the left atrial appendage 24. A second artificially created opening 56 may be formed through a wall 58 of the pulmonary vein 20. The first and second artificially created openings 52 and 56, which may be formed using any known artificial or man-made means, may be joined together at the anastomosis 50. According to one example, sutures 60 may be used to secure the anastomosis 50. It should be appreciated that the first and second artificially created openings 52 and 56 may be formed in any order or may be formed simultaneously.

The left atrial appendage shunt 32 includes a circulation conduit 62 fluidly connecting the left atrial appendage 24 and the pulmonary vein 20. According to the exemplary embodiment, the circulation conduit 62 may be defined by a portion of the wall 54 of the left atrial appendage 24 defining the first artificially created opening 52 and a portion of the wall 58 of the pulmonary vein 20 defining the second artificially created opening 56. The circulation conduit 62, which may vary greatly in configuration depending on the particular application, includes structures fluidly connecting the interior 26 of the left atrial appendage 24 and the interior 34 of the pulmonary vein 20. The circulation conduit 62 has a first end 64 opening at, or defined by, the first artificially created opening 52 and a second end 66 opening at, or defined by, the second artificially created opening 56.

The direct anastomosis 50 may alternatively be formed using other known means, including the use of tissue fusing, adhesives, magnets, or other bonding or pressure application devices. For example, as shown in FIG. 3, magnets 68 may be placed in the left atrial appendage 24 and the pulmonary vein 20 that are attracted to one another. Over time, the tissue captured between the magnets 68 will necrose, while the tissue surrounding the magnets 68 will join together forming a pathway when the magnets 68 are removed. As a result, the first and second artificially created openings 52 and 56 and the circulation conduit 62 may be formed. A magnet delivery system for forming such an anastomosis is described in U.S. Patent Application Publication 2010/0292729 to Aguirre et al.

Alternatively, as shown in FIG. 4, a vessel 70 may define the circulation conduit 62, which fluidly connects the left atrial appendage 24 with the pulmonary vein 20. In particular, an open first end 72 of the vessel 70 may be attached to the left atrial appendage 24 such that the open first end 72 is in fluid communication with the first artificially created opening 52 through the left atrial appendage 24. An open second end 74 of the vessel 70 may be attached to the pulmonary vein 20 such that the open second end 74 is in fluid communication with the second artificially created opening 56 of the left atrial appendage 24. As should be appreciated, the open first end 72 of the vessel 70 represents the first end 64 of the circulation conduit 62, while the open second end 74 of the vessel 70 represents the second end 66 of the circulation conduit 62. The vessel 70 may be surgically attached, for example, using known medical attachment means. According to some examples, the vessel 70 may include a homograft vessel, autograft vessel, bioprosthetic vessel, and/or may be made from a synthetic material.

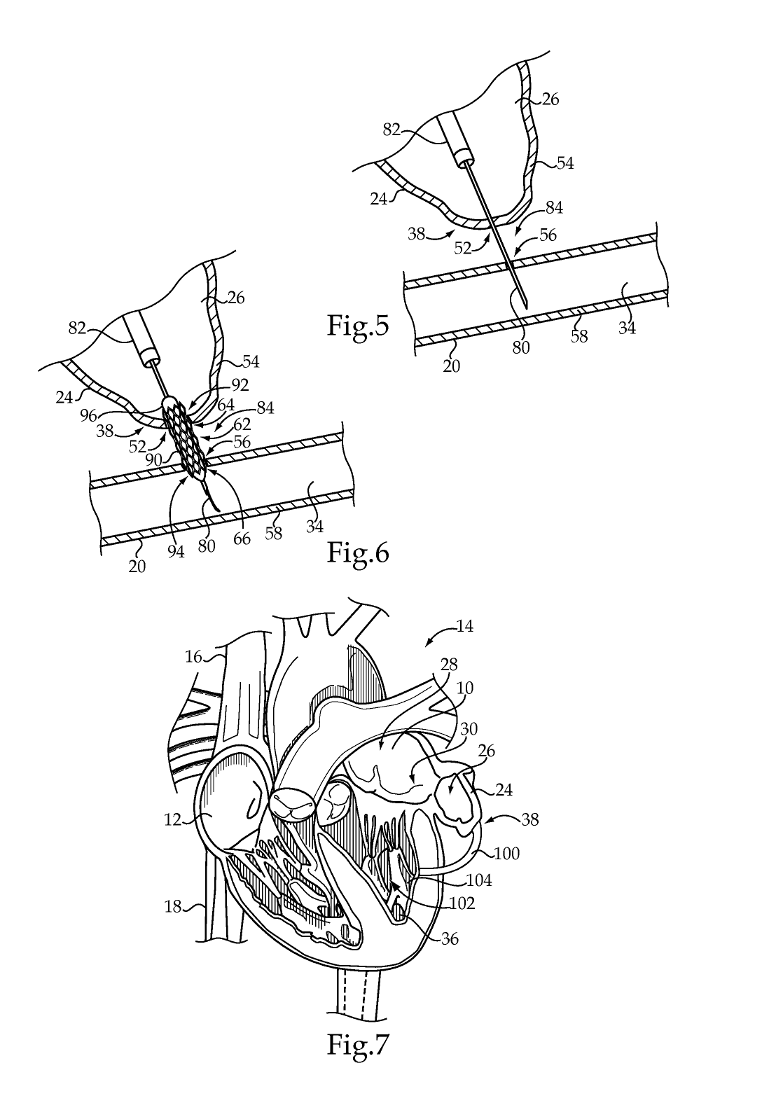

Yet alternatively, the left atrial appendage shunt 32 may be formed using a stent placement procedure. For example, as shown in FIG. 5, a needle 80, which may be advanced into the left atrial appendage 24 through a delivery catheter 82, may puncture through the wall 54 of the left atrial appendage 24 to form the first artificially created opening 52. Thereafter, the needle 80 may be advanced along a passage 84 between the left atrial appendage 24 and the pulmonary vein 20 and may puncture through the wall 58 of the pulmonary vein 20 to form the second artificially created opening 56. Referring now to FIG. 6, and at a later stage of the stent placement procedure, a stent 90 may be positioned within the first artificially created opening 52, the passage 84, and the second artificially created opening 56 to define the circulation conduit 62. A first end 92 of the stent 90 may represent the first end 64 of the circulation conduit 62, while a second end 94 of the stent 90 may represent the second end 66 of the circulation conduit 62. A balloon 96 may be used to expand the stent 90 and/or may be inflated to dilate the passage 84, which may include intervening tissue between the left atrial appendage 24 and the pulmonary vein 20, before or after placement of the stent 90.

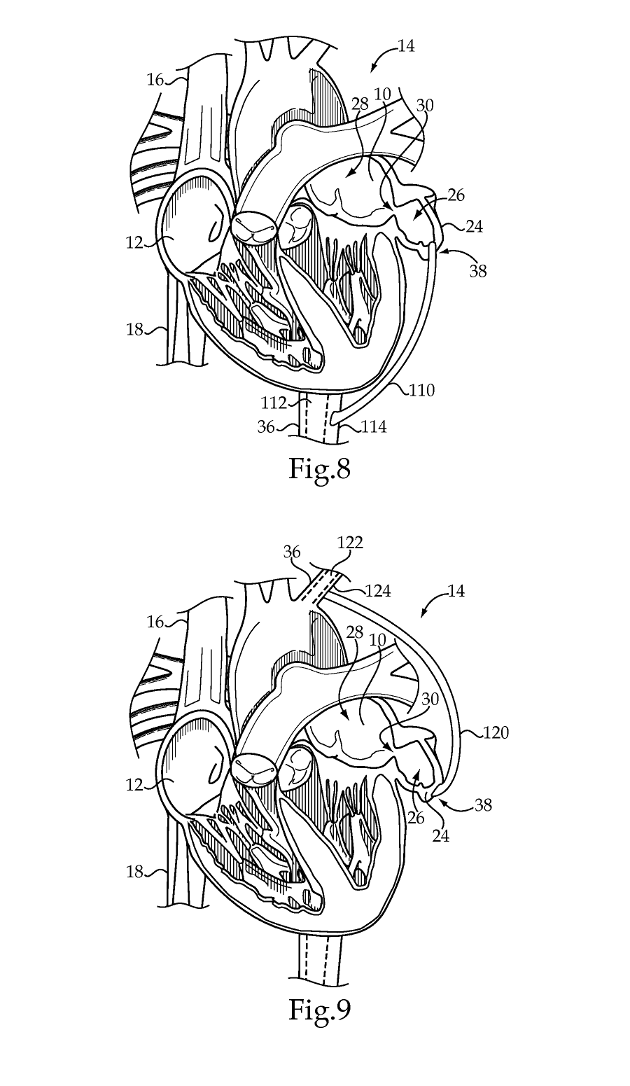

An alternative shunt for reducing blood clot formation in the left atrial appendage 24 is shown generally at 100 in FIG. 7. In particular, the left atrial appendage shunt 100 may be configured to fluidly connect the interior 26 of the left atrial appendage 24 and an interior 102 of a left ventricle 104. As shown, the left atrial appendage shunt 100 is distinct from the blood flow passage provided by the ostium 30 of the left atrial appendage 24. Alternatively, as shown in FIG. 8, a left atrial appendage shunt 110 may be configured to fluidly connect the interior 26 of the left atrial appendage 24 and an interior 112 of a descending aorta 114. Yet alternatively, as shown in FIG. 9, a left atrial appendage shunt 120 may be configured to fluidly connect the interior 26 of the left atrial appendage 24 and an interior 122 of a subclavian artery 124. These alternative shunts may also be formed using the methods described above and/or alternative methods.

It should be appreciated that the above disclosed shunts 32, 100, 110, and 120 are provided for exemplary purposes only, and the target cardiovascular structure 36 may include a variety of different cardiovascular structures. The shunts 32, 100, 110, and 120, and alternative shunts, may be artificially created using the methods disclosed herein and/or additional known medical processes for creating shunts. It should be appreciated that the configuration of the shunt 32, 100, 110, and 120 may be depend on the location and configuration of the target cardiovascular structure 36. Further, it should be appreciated that the target cardiovascular structure 36 may be selected based on location, accessibility, and pressure and/or flow considerations. Depending on the particular target cardiovascular structure 36, a valve positioned along the circulation conduit 62 may be desired.

### Industrial Applicability

The present disclosure is generally applicable to means for inducing blood flow through the left atrial appendage and, thus, reducing blood clot formation in the left atrial appendage. More specifically, the present disclosure is applicable to a left atrial appendage shunt, independent from the ostium of the left atrial appendage, for fluidly connecting the left atrial appendage and a target cardiovascular structure. According to some examples, the target cardiovascular structure may include a pulmonary vein, the left ventricle, the descending aorta, or a subclavian artery.

Referring generally to FIGS. 1-9, a left atrial appendage 24 is a muscular pouch connected to a left atrium 10 of a heart 14 that serves as a reservoir for the left atrium 10. In patients with atrial fibrillation, the contractions of the left atrium 10 and the right atrium 12 are not properly synchronized with the contractions of the left ventricle and the right ventricle. When this occurs, the left atrial appendage 24 may not contract regularly with the left atrium 10 and, as a result, blood may pool in the left atrial appendage 24. In addition, the left atrial appendage 24 is a highly trabeculated structure that provides flow resistant voids where blood is more likely to pool. To reduce blood pooling for these patients and the potential for blood clot formation, a left atrial appendage shunt 32, 100, 110, and 120, as disclosed herein, may be formed. In particular, the left atrial appendage shunt 32, 100, 110, and 120 may induce blood flow through the left atrial appendage 24. Preferably, the left atrial appendage shunt 32, 100, 110, and 120 may open at a distal tip 38 of the left atrial appendage 24 to induce blood flow throughout the left atrial appendage 24.

The left atrial appendage shunt 32, 100, 110, and 120 between the left atrial appendage 24 and another part of the heart 14 or a blood vessel will permit blood to continually flow through the appendage 24, thus reducing the occurrences of the blood becoming stagnant and forming clots. As described above, the particular target cardiovascular structure 36, along with the means for creating the shunt 32, 100, 110, and 120, and the resulting configuration of the shunt 32, 100, 110, and 120 may vary greatly. The shunt formation described herein is an alternative to current treatment options, which include the administration of blood thinning medications, occlusion of the left atrial appendage 24, and removal of the left atrial appendage 24. Occlusion devices may be traumatic to the left atrial appendage 24, may become dislodged, may lead to atrioventricular block, and/or may fail to completely occlude the left atrial appendage 24. Further, occluding or removing the left atrial appendage 24 reduces the overall volume of the left atrium 10 and, thus, may adversely affect the pressure within the atrium 10. Thus, the shunting, as described herein, spares the reservoir function of the left atrial appendage, which is not the case with the current treatment options.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A left atrial appendage shunt for reducing blood clot formation in a left atrial appendage, comprising:
a first artificially created opening through a wall of the left atrial appendage;
a second artificially created opening through a wall of a target cardiovascular structure; and
a circulation conduit fluidly connecting an interior of the left atrial appendage and an interior of the target cardiovascular structure, and having a first end opening at the first artificially created opening and a second end opening at the second artificially created opening.

2. The left atrial appendage shunt of claim 1, wherein the first and second artificially created openings are joined at a direct anastomosis.

3. The left atrial appendage shunt of claim 1, wherein the circulation conduit is defined by a portion of the wall of the left atrial appendage defining the first artificially created opening and a portion of the wall of the target cardiovascular structure defining the second artificially created opening.

4. The left atrial appendage shunt of claim 1, wherein the circulation conduit includes a homograft vessel.

5. The left atrial appendage shunt of claim 1, wherein the circulation conduit includes an autograft vessel.

6. The left atrial appendage shunt of claim 1, wherein the circulation conduit includes a bioprosthetic vessel.

7. The left atrial appendage shunt of claim 1, wherein the circulation conduit is made from a synthetic material.

8. The left atrial appendage shunt of claim 7, wherein the circulation conduit includes a stent.

9. The left atrial appendage shunt of claim 1, wherein the target cardiovascular structure is a pulmonary vein.

10. The left atrial appendage shunt of claim 1, wherein the target cardiovascular structure is a left ventricle.

11. The left atrial appendage shunt of claim 1, wherein the target cardiovascular structure is a descending aorta.

12. The left atrial appendage shunt of claim 1, wherein the target cardiovascular structure is a subclavian artery.

13. The left atrial appendage shunt of claim 1, wherein the first artificially created opening is formed through a distal tip of the left atrial appendage.

14. A method of reducing blood clot formation in a left atrial appendage using a left atrial appendage shunt, the method comprising:
forming a first artificially created opening through a wall of the left atrial appendage;
forming a second artificially created opening through a wall of a target cardiovascular structure; and
fluidly connecting an interior of the left atrial appendage and an interior of the target cardiovascular structure with a circulation conduit having a first end opening at the first artificially created opening and a second end opening at the second artificially created opening.

15. The method of claim 14, wherein the fluidly connecting step includes joining the first and second artificially created openings at a direct anastomosis.

16. The method of claim 14, wherein the fluidly connecting step includes:
attaching an open first end of a vessel to the left atrial appendage such that the open first end is in fluid communication with the first artificially created opening; and
attaching an open second end of the vessel to the target cardiovascular structure such that the open second end is in fluid communication with the second artificially created opening.

17. The method of claim 14, wherein the fluidly connecting step includes:
advancing a needle through the wall of the left atrial appendage to form the first artificially created opening, along a passage between the left atrial appendage and the target cardiovascular structure, and through the wall of the target cardiovascular structure to form the second artificially created opening; and
positioning a stent within the first artificially created opening, the passage, and the second artificially created opening.

18. The method of claim 17, further including advancing the needle through a catheter and into the left atrial appendage prior to the puncturing step.

19. The method of claim 17, further including inflating a balloon within the passage.

20. The method of claim 14, wherein the target cardiovascular structure is a pulmonary vein.
